(19) United States
(12) Patent Application Publication
(10) Pub. No.: US 2020/0008777 A1
(43) Pub. Date: Jan. 9, 2020

(54) METHOD FOR OCULAR ULTRASOUND WITH ANNULAR TRANSDUCERS

(71) Applicant: QUANTEL MEDICAL, COURNON-D'AUVERGNE (FR)

(72) Inventors: Christian CHABRIER, LA ROCHE BLANCHE (FR); Cédric VENUAT, CLERMONT FERRAND (FR)

(73) Assignee: QUANTEL MEDICAL, COURNON-D'AUVERGNE (FR)

(21) Appl. No.: 16/487,693

(22) PCT Filed: Feb. 21, 2018
(86) PCT No.: PCT/FR2018/050405

§ 371 Date: Aug. 21, 2019

(30) Foreign Application Priority Data
Feb. 22, 2017 (FR) 1751386

(51) Int. Cl. A61B 8/10; A61B 8/00

(52) U.S. Cl. CPC A61B 8/10; A61B 8/5207; A61B 8/429; A61B 8/4494

(57) ABSTRACT

The invention relates to a method for ocular ultrasound using an ultrasound probe comprising a plurality of transducer elements organised in at least n concentric rings forming n transducer rings (2, 2a, 2b, 2c, 2d, 2e), the transducer rings being grouped together in k groups of rings. Said method comprises the following steps: for each cycle (100, 110, 200, 210, 300, 310) of a plurality of cycles of k iterations using a different group of transducer rings and working through the groups of transducer rings: exciting a group of transducer rings in order to emit ultrasound waves; collecting n measuring signals from the n transducer rings; combining the n measuring signals in order to provide an ultrasound line representing the response of the n transducer rings when ultrasound waves are emitted; combining k ultrasound lines resulting from the most recent k iterations into a displayable line; and treating and displaying the displayable lines.

### TECHNICAL FIELD OF THE INVENTION

The present invention belongs to the field of ophthalmology. More specifically, the invention relates to an ophthalmic echography method using annular transducers.

Ophthalmic echography consists in the acquisition of an eye image by means of an ultrasonic probe emitting an ultrasound beam. The ultrasonic probe is positioned in the vicinity of an eye, typically by being placed in contact with the eye, and emits ultrasounds propagating through the eye. These ultrasounds pass through the internal structures of the eye, such as the crystalline lens, the vitreous body or the retina, and are partly reflected by these structures. The reflected ultrasounds are captured and recorded by the probe to give images of the eye. The propagation of the ultrasonic waves depends not only on their frequency, but very much on the spatial configuration of the ultrasonic probe that emits them.

The acquisition of ultrasonic images operable for the most part of an eye complies with many constraints with the conventional transducers. For example, in order to examine a complete eye and its socket with a mechanically oscillating ultrasonic probe and obtain the best possible image, it is necessary to emit waves in frequencies ranging from 10 to 25 Mhz, so as to reach a depth of more than 45 mm in the eye (between 45 and 60 mm). It is also necessary to vary the emission angle from 45 to 60°, to focus the transducer to about 25 mm (between 18 and 27 mm) just before or on the retina (which constitutes the preferred target), and to guarantee an image frequency of 8 hz minimum (between 8 and 16 hz) to visualize the movements of the vitreous body.

By using a curved single-element transducer with a diameter of 9 mm (maximum diameter to have 50° of displacement), it is for example possible to obtain at the natural focal length given by the curvature of the transducer, at an ultrasound frequency of 20 MHz (with a bandwidth ranging from 10 to 30 MHz), a total longitudinal resolution of 75 μm, and a total possible lateral resolution of 200 μm. However, the image quickly becomes blurred with the depth since the depth of field at 6 dB is then only of 2.5 mm, which means that only a small area around the retina will have a signal-to-noise ratio and an optimum resolution. Furthermore, in accordance with the Shannon's theorem, it is necessary to sample at least twice the highest frequency of the bandwidth to be processed (30 MHz for a central frequency transducer of 20 MHz), namely at 60 MHz and to generate enough line in order to be at least 2.5 times the total lateral resolution at the focal length namely 360 lines minimum for 50°.

Regarding the ultrasound emission itself, it is necessary to wait for the end of the echo signal (i.e. the reflected ultrasounds) of an echographic line to send back another one, in order to avoid the clutter of one line on the other. There is then an echographic line at best every 90 μs for a depth of 60 mm, which results in a maximum probe speed of about 13 hz. However, by accepting certain compromises on the quality of the acquired echographic images, it is possible to reach image frequencies of up to 20 Hz.

In order to allow an improved resolution and eye penetration, it has been proposed to use an annular transducer, i.e., a transducer comprising a plurality of transducer elements organized in concentric rings forming transducer rings. The use of an annular transducer allows increasing the depth of fields to have the best possible overall image of the eye whatever its geometry (small, large, oval . . . ).

By resuming the configuration of the example of single-element transducer mentioned above (ultrasonic waves at 20 MHz and a diameter of 9 mm), it is possible to obtain, with a curved transducer having five ultrasonic rings, a depth of field at 6 dB of 18 mm, namely an approximately 7-fold improvement. The use of a transducer of this type therefore considerably improves the quality of the vitreous and retinal image of the eye. In addition, it also improves the signal-to-noise ratio over the entire depth of fields.

Moreover, ultrasonic waves at higher frequencies ranging from 35 to 50 MHz can be used to increase accuracy when examining the overall anterior pole of an eye. A conventional curved single-element transducer, focused at a depth of about 10 mm, then allows examination up to 16 mm deep. The depth of field obtained is then of about 1 mm, which is very unsatisfactory. By using a five-ring annular transducer, a depth of fields of 8 mm is reached, which makes the image better as a whole and the use of this configuration much simpler because of its reduced sensitivity to the depth adjustment which must be performed by the operator.

However, the use of several concentric rings requires the coordination of emission and reception of the measurement signals between these rings. Two approaches have been used so far.

A first approach is similar to the operation of the phased-array transducers. Emission is made on the whole set of the transducer rings with an emission time delay calculated between each transducer ring so that the echographic ultrasonic waves arrive in phase at a certain depth. The echographic lines obtained by the reception of the ultrasonic echoes by the set of the transducer rings are then added in real time. A high-quality image is thus obtained around the target depth, and the speed is comparable to that of a single-element transducer.

For example, the patent application US 2005/251043 A1 describes a method for exploring and visualizing tissues of human or animal origin, in which:
an ultrasonic probe carried by a driven head is positioned via a three-dimensional positioning system, in particular controlled by a computer in line with said tissue structure,
the probe is controlled such that it generates beams of high-frequency converging ultrasonic waves (ranging from 30 to 50 MHz), these waves being focused at a given area of tissue structure, with a penetration distance comprised between 20 and 30 mm,
a scanning of the tissue structure is carried out by the computer-driven positioning system, by performing in parallel an acquisition, by the computer, of the signals reflected by the tissue structure,
various signal processing operations are performed on the data derived from the scanning, to improve the return of information and facilitate the interpretation by the practitioner.

In this patent application US 2005/251043 A1, a dynamically-focused probe is used made by an electronic or digital control method, composed of a multi-element probe with a circular symmetry, composed of several concentric annular transducers evenly spaced on a flat surface or a surface with spherical concavity. These transducers are independent of each other and are individually controlled at emission and at reception by time-offset pulses. Particularly, a dynamic focusing is obtained by introducing a phase-shift/time delay at emission between the different rings. The set of the transducer rings emit with a calculated emission time limit between each transducer ring so that the echographic ultrasonic waves arrive in phase at a certain depth. The echographic lines obtained by the reception of the ultrasonic echoes by the set of the transducer rings are then added in real time.

However, when it is desirable to obtain an overall quality image of an eye, several depths must be targeted. It is therefore necessary to make several passages by modifying the emission delays affecting each transducer ring to reach different depths. This results in a very long acquisition time since the acquisition frequency is divided by the number of different depths to be analyzed in order to reconstruct the overall image.

A second approach is similar to the operation of the radars. A single transducer ring is excited and emits ultrasonic waves. On the other hand, the signals of all the transducer rings, resulting from the reception by these transducer rings of the reflected ultrasonic waves, are recovered and adjusted (to compensate for the path difference between transducer rings). In order to obtain the final image, it is however necessary to use all the transducer rings in emission, which therefore requires as many emission-reception iterations as the number of transducer rings. Thus, in one example with five transducer rings, this means carrying out five successive emissions, with five receptions per emission, namely a total of 25 partial echographic lines that must be processed to give an overall echographic line. Consequently, this approach requires five times longer than a single-element transducer. Yet, speed is important in ophthalmic echography in order to be able to observe the movements of the vitreous body.

Moreover, this approach requires a significant post-processing because of the numerous partial echographic lines, thereby implying a non-negligible calculation time, which may require management of the offsets per timeslot. In addition, each emission being made only on a transducer ring, the final result of the signal-to-noise ratio at the focal length is similar to that of a single-element transducer but 6 dB lower than the previous method. US Patent Application 2013/0093901 A1 uses this approach, and in order to accelerate the image acquisition, proposes not to use certain lines, which leads to a lower image quality in terms of resolution, sensitivity and penetration.

### PRESENTATION OF THE INVENTION

The object of the invention is to propose an ocular echography method using an ultrasonic probe with several transducer rings making it possible to rapidly acquire good quality images.

For this purpose, it is proposed an ocular echography method using an ultrasonic probe including a plurality of transducer elements organized into at least n concentric rings forming n transducer rings, the transducer rings being grouped into several groups of rings each grouping between 1 and n−1 transducer rings, each group of rings being differentiated from another group of transducer rings by at least one transducer ring different from the transducer rings of said other group of transducer rings, the groups of rings being equal to a number k, the method comprising the following steps:
a) for each cycle of a plurality of cycles of k iterations, each iteration involving a group of different transducer rings, each cycle running through the set of the k groups of transducer rings, during each iteration:
   a1) exciting a group of transducer rings so that the transducer rings of said group of transducer rings emit ultrasonic waves at an emission frequency;
   a2) recovering n measurement signals from the n transducer rings, each measurement signal resulting from the reception by a transducer ring of reflected ultrasonic waves resulting from the emission of ultrasonic waves by said group of transducer rings;
   a3) combining the n measurement signals to give an echographic line, said echographic line being representative of the response of then transducer rings to the emission of ultrasonic waves by said group of ultrasonic rings;
b) combining into a displayable line of k echographic lines resulting from the most recent k iterations;
c) processing and displaying the displayable lines.

The method is advantageously completed by the following characteristics, taken alone or in any one of their technically possible combination:
in each cycle, the iterations are performed in the same order;
the measurement signal of a transducer ring results from the digitization of the reception signal generated by this transducer ring upon reception by said transducer ring of reflected ultrasonic waves resulting from the emission of ultrasonic waves by a group of transducer rings, a measurement signal being defined as a chronological sequence of discrete points with which corresponding values are associated, and the combination of measurement signals to give an echographic line during an iteration consists in adding the values associated with synchronous discrete points of said measurement signals;
the combination of measurement signals to give an echographic line during an iteration is restricted to a selection of discrete points, said discrete points being selected so as to make a chronological offset between the measurement signals compensating for the acoustic path differences resulting from the geometry of the transducer, the synchronism of the discrete points taking into account this offset;
discrete points are added to the measurement signal by convolving said measurement signal with a sliding cardinal sine function so that a period between the discrete points is less than the inverse of at least ten times the emission frequency;
an echographic line is defined as a chronological sequence of discrete points with which corresponding values are associated, and wherein the combination of echographic lines to give a displayable line consists in adding the values associated with synchronous discrete points of said echographic lines;
the transducer rings are grouped into k groups of rings, each grouping between 2 and n−1 transducer rings, with 1<k<n;
n=5, the ultrasonic probe comprising five transducer rings.

The invention also relates to a computer program product comprising program code instructions recorded on a non-volatile medium that can be used in a computer for performing the steps of processing the method according to the invention, when said program is run on a computer.

The invention also relates to an echography system comprising an ultrasonic probe including a plurality of transducer elements organized into at least n concentric rings forming n transducer rings, a unit for processing said ultrasonic probe and a screen, said processing unit being configured to implement the method according to the invention, the transducer rings being grouped into several groups of rings each grouping between 1 and n−1 transducer rings, each group of rings being differentiated from another group of transducer rings by at least one transducer ring different from the transducer rings of said other group of transducer rings, the groups of rings being equal to a number k, the processing unit being configured to:
a) for each cycle of a plurality of cycles of k iterations, each iteration involving a different group of transducer rings, each cycle running through the set of the k groups of transducer rings, during each iteration:
excite a group of transducer rings so that the transducer rings of said group of transducer rings emit ultrasonic waves at an emission frequency;
recover n measurement signals from the n transducer rings, each measurement signal resulting from the reception by a transducer ring of reflected ultrasonic waves resulting from the emission of ultrasonic waves by said group of transducer rings;
combine the n measurement signals to give an echographic line, said echographic line being representative of the response of the n transducer rings to the emission of ultrasonic waves by said group of ultrasonic rings;
b) combine into a displayable line of k echographic lines resulting from the most recent k iterations;
c) process and send the displayable lines to the screen.

### PRESENTATION OF THE FIGURES

The invention will be better understood, thanks to the following description, which refers to embodiments and variants according to the present invention, given as non-limiting examples and explained with reference to the appended schematic drawings, wherein:

FIG. 1 schematically illustrates the annular configuration of the transducer elements of an ultrasonic probe according to one possible embodiment of the invention,

FIGS. 2, 3 and 4 schematically illustrate the course of examples of the echography method according to different embodiments of the invention.

### DETAILED DESCRIPTION

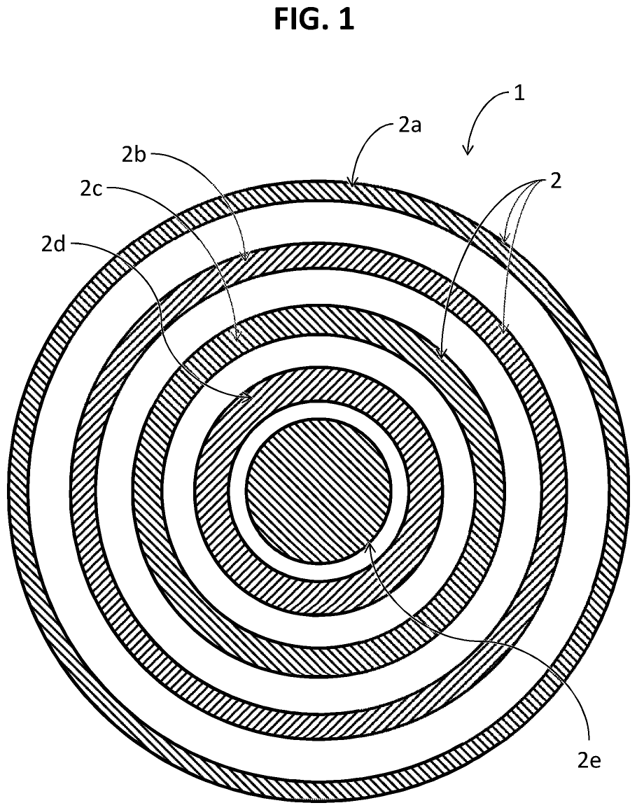

Referring to FIG. 1, an ultrasonic probe 1 is used, including a plurality of transducer elements organized in n concentric rings 2 forming n transducer rings. In the example of FIG. 1, there are five transducer rings, designated respectively from outside to the center by 2a, 2b, 2c, 2d and 2e, and consequently n=5. It is understood, however, that n can take other values. However, the number of transducer rings 2 is greater than three (i.e. n>3), and preferably greater than four (i.e. n>4). The transducer elements are for example piezoelectric, configured to emit ultrasounds propagating into the eye. These ultrasounds typically have a frequency comprised between 10 and 100 MHz. It should be noted here that the central transducer ring 2e is solid, and thereby constitutes a transducer disc. The central transducer ring 2e could be hollow as well, that is to say with an empty center like the other peripheral transducer rings 2a, 2b, 2c and 2d. The different transducer rings being concentric, it is necessary that the peripheral transducer rings 2a, 2b, 2c and 2d are hollow so that they can be nested within each other. There is no such need for the central transducer ring 2e, which can therefore be solid.

The natural focal length of the ultrasonic probe 1 is given by the curvature of the transducer elements or by the addition of a lens facing its emission face. For the ocular examination, this curvature can vary from flat to radius of curvature of 9 mm, for example. The largest transducer ring 2a has an external diameter comprised between 3 and 10 mm, for example 9 mm, and has a width of 0.05 mm. The smallest transducer ring 2e has an external diameter comprised between 0.1 and 0.3 mm, for example 0.2 mm. The transducer rings 2 are separated by a distance comprised between 0.02 and 0.1 mm, for example 0.05. Preferably, in order for the transducer rings to have an equivalent power therebetween, it may be sought to make their respective surfaces similar, and ideally identical, in size. For this purpose, the width of the transducer rings decreases preferably with their distance to the common center.

For the implementation of the ophthalmic echography method, the ultrasonic probe 1 is positioned relative to an eye, in order to be able to emit and receive ultrasonic waves propagating inside this eye. The ultrasonic probe 1 can be brought into contact with the eye, that is to say, contiguous to the cornea or to the sclera, possibly covered with a gel. It is also possible to provide the presence of a pocket of liquid such as water between the ultrasonic probe 1 and the eye, this pocket may be typically formed by a permanently closed membrane on the ultrasonic probe 1. The ultrasonic probe 1 can also be immersed in a liquid contained in a cup opened against the eye, the liquid serving as intermediate propagation medium between the ultrasonic probe 1 and the eye.

Once positioned, the ultrasonic probe 1 is controlled to emit and receive ultrasonic waves. Each transducer ring 2 is individually controlled, and in response to an excitation (an electrical voltage signal), a transducer ring 2 emits ultrasonic waves at an emission frequency. The emission frequency is comprised between 10 and 100 MHz. In the following example, an emission frequency of 20 MHz will be described.

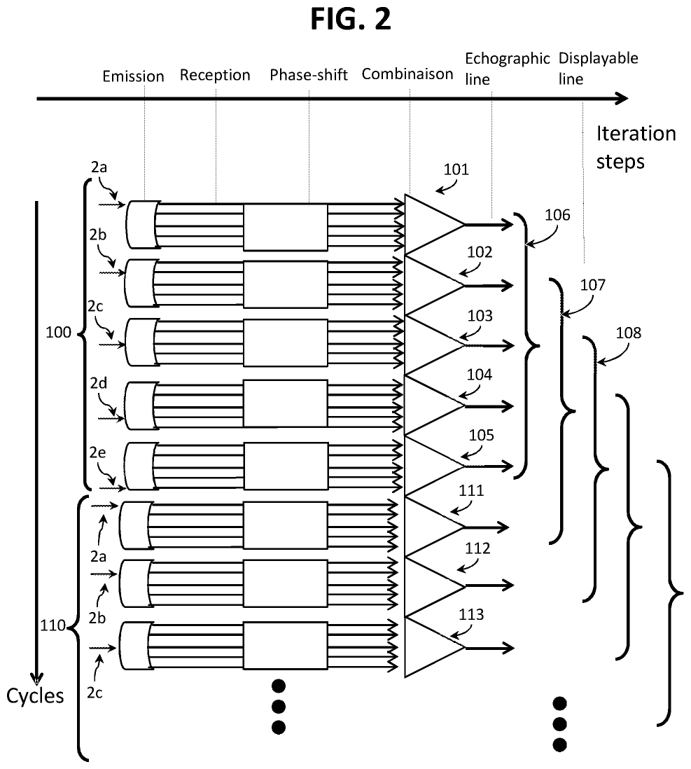

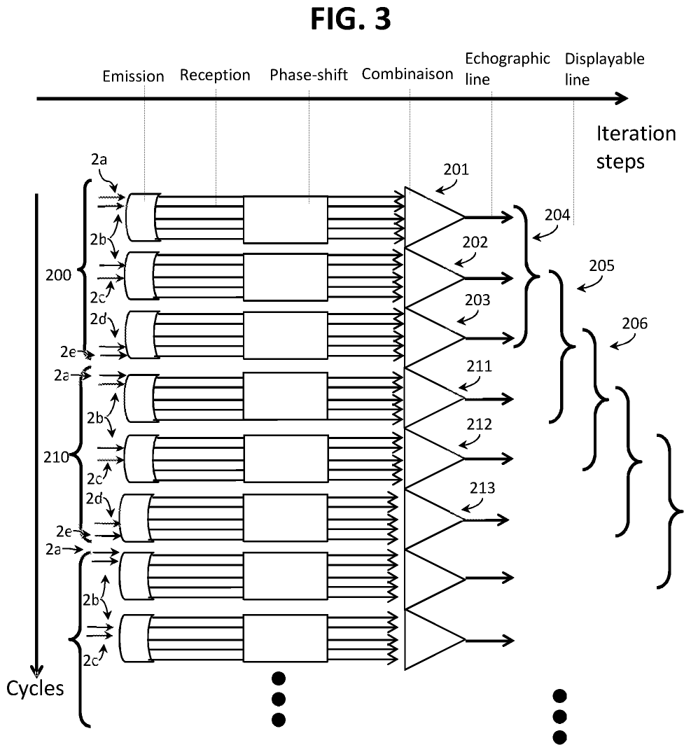

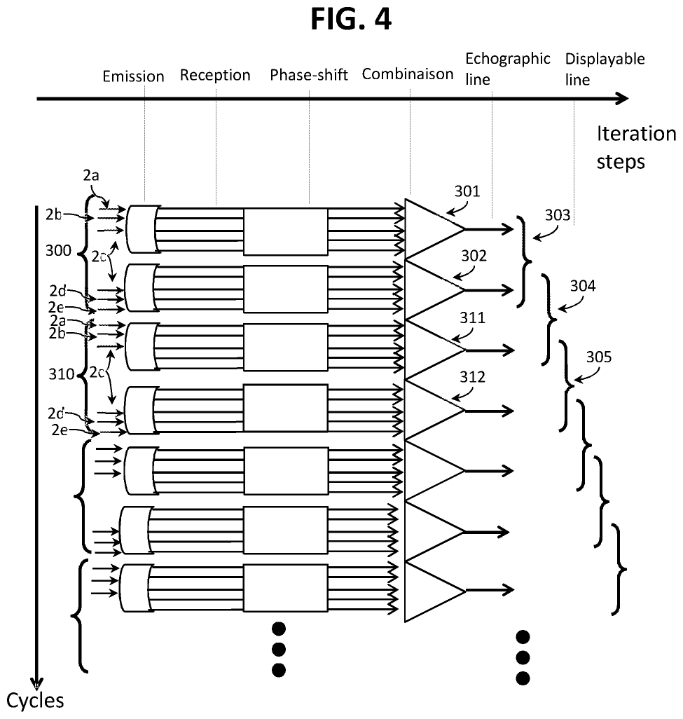

In the context of the method, the transducer rings 2 are grouped into several groups of rings grouping between 1 and n−1 transducer rings. For example, if n=5, then each group of rings can group one, two, three or four transducer rings 2. The number of ring groups is k, with l≤k≤n. Preferably, k is at least equal to two, and preferably l<k≤n, also preferably, l<k<n. The transducer rings are consequently grouped into several groups of rings (k>2). Thus, in the example of FIG. 2, n=5 and k=5, in the example of FIG. 3, n=5 and k=3, in the example of FIG. 4, n=5 and k=2.

In order to ensure equivalence between the groups, the different groups of transducer rings preferably have the same number of transducer rings 2. A transducer ring 2 can be part of two groups of rings. However, each group of transducer rings is differentiated from another group of transducer rings by at least one transducer ring 2 different from the transducer rings 2 of said other group of transducer rings. Preferably, each group of transducer rings comprises at least one transducer ring 2 belonging to no other group of transducer rings.

For example, in the case of FIG. 3, a first group of transducer rings groups the transducer rings 2a and 2b, a second group of transducer rings groups the transducer rings 2b and 2c, and the third group of transducer rings groups the transducer rings 2d and 2e. It is thus found that the transducer ring 2b is part of two groups of transducer rings, but also that the transducer ring 2a is part of the only first group of rings and no other group of rings, that the transducer ring 2c is part of the only third group of rings and no other group of rings, and that the transducer rings 2c and 2d are part of the only third group and no other group of rings.

These groupings in different groups result in an emission control common to the transducer rings 2 of the same group of rings at an emission instant. It is possible to modify the number or the composition of the groups of transducer rings from one implementation of the method to another. It should be noted that it is not necessary to select all the transducer rings 2 of the ultrasonic probe 1 to emit ultrasounds. In some configurations, transducer rings 2 may not emit ultrasounds and be used only in reception.

The method comprises a plurality of cycles. A cycle comprises several iterations of emission-reception of ultrasonic waves, each time involving a different group of transducer rings. Each cycle runs through the whole set of the k groups of transducer rings. During each iteration:
a group of transducer rings is excited so that only the transducer rings 2 of said group of transducer rings emit ultrasonic waves at an emission frequency, whereas the other transducer rings 2, not part of said group of transducer rings, do not emit ultrasonic waves at the emission frequency, and
the n measurement signals of the set of the n transducer rings are recovered, each measurement signal resulting from the reception by a transducer ring 2 of reflected ultrasonic waves resulting from the emission of ultrasonic waves by the transducer rings or the group of transducer rings,
the n measurement signals are combined to give an echographic line.

Preferably, in each cycle, the iterations are performed in the same order. The iterations of each cycle allow determining and displaying a displayable line. There are therefore at least as many cycle iterations as there are lines in the displayed image. However, between each cycle, each image or group of images, the modes of emission of the ultrasonic waves can be modified to promote the resolution (individual emissions), the penetration (emission on grouped rings) or the speed (non-use of all the rings), knowing that all combinations are possible on each image and possibly on each line in terms of emission, grouping of rings and number of rings used in emission or reception.

### Emission

In the example of FIG. 2, each group of transducer rings comprises only one single ultrasonic transducer ring 2. The first group of rings consists of the ultrasonic transducer ring 2a, the second group of rings consists of the ultrasonic transducer ring 2b, the third group of rings consists of the ultrasonic transducer ring 2c, the fourth group of rings consists of the ultrasonic transducer ring 2d, and the fifth group of rings consists of the ultrasonic transducer ring 2e. There are therefore five groups of rings (k=5) for five transducer rings (n=5).

A first cycle 100 comprises five iterations 101, 102, 103, 104 and 105. In a first iteration 101, only the transducer ring 2a of the first group of rings is excited by an electrical control signal and emits ultrasonic waves. In a second iteration 102, only the transducer ring 2b of the second group of rings is excited by an electrical control signal and emits ultrasonic waves. In a third iteration 103, only the transducer ring 2c of the third group of rings is excited by an electrical control signal and emits ultrasonic waves. In a fourth iteration 104, only the transducer ring 2d of the fourth group of rings is excited by an electrical control signal and emits ultrasonic waves. In a fifth iteration 105, only the transducer ring 2e of the fifth group of rings is excited by an electrical control signal and emits ultrasonic waves.

Once this first cycle 100 is completed, that is to say when said first cycle 100 has run through the set of five groups of transducer rings 101, 102, 103, 104, 105, a second cycle 110 then begins, running through the set of five groups of transducer rings during five iterations 111, 112, 113 in the same way as the first cycle 100.

In the example of FIG. 3, each group of transducer rings consists of two ultrasonic transducer rings. The first group of rings consists of the ultrasonic transducer rings 2a and 2b, the second group of rings consists of the ultrasonic transducer rings 2b and 2c, the third group of rings consists of the ultrasonic transducer rings 2d and 2e. There are therefore three groups of rings (k=3) for five transducer rings (n=5).

A first cycle 200 comprises three iterations 201, 202, 203. In a first iteration 201, only the transducer rings 2a and 2b of the first group of rings are excited by an electrical control signal and emit ultrasonic waves. In a second iteration 202, only the transducer rings 2b and 2c of the second group of rings are excited by an electrical control signal and emit ultrasonic waves. In a third iteration 203, only the transducer rings 2d and 2e of the third group of rings are excited by an electrical control signal and emit ultrasonic waves. Once this first cycle 200 is over, that is to say when said first cycle 200 has run through the set of three groups of transducer rings, a second cycle 210 then begins, running through the set of three groups of transducer rings during three iterations 211, 212, 213 in the same way as the first cycle 200.

In the example of FIG. 4, there are two groups of transducer rings, and each group of transducer rings consists of three transducer rings. The first group consists of the ultrasonic transducer rings 2a, 2b and 2c, the second group consists of the ultrasonic transducer rings 2c, 2d, and 2e. There are therefore two groups of rings (k=2) for five transducer rings (n=5).

A first cycle 300 comprises two iterations 301, 302. In a first iteration 301, only the three transducer rings 2a, 2b, and 2c of the first group of rings are excited by an electrical control signal and emit ultrasonic waves. In a second iteration 302, only the three transducer rings 2c, 2d, and 2e of the second group of rings are excited by an electrical control signal and emit ultrasonic waves. Once this first cycle 300 is over, that is to say when said first cycle 300 has travelled through the set of the two groups of transducer rings 301, 302, a second cycle 310 then begins, travelling through the set of the two groups of transducer rings during two iterations 311, 312, in the same way as the first cycle 300.

There is therefore, for each iteration, an emission of ultrasonic waves, a reception of ultrasonic waves, and the combination of measurement signals resulting from this reception.

### Reception

The emitted ultrasounds propagate into the eye, pass through the internal structures of the eye, such as the crystalline lens, the vitreous body or the retina, and are partly reflected by these structures and sent back to the ultrasonic probe 1. The reception, by a transducer ring 2, of reflected ultrasonic waves generates a measurement signal. All the transducer rings 2 receive these reflected ultrasonic waves and generate measurement signals. Thus, in the case where the ultrasonic probe comprises five transducer rings 2, five measurement signals are generated and used.

More specifically, the reception of the ultrasonic waves by a transducer ring 2 causes the occurrence of an electrical and analog reception signal, at the output of said transducer ring 2. This reception signal is then digitized to give a measurement signal. A measurement signal is therefore a digital signal and is defined as a chronological sequence of discrete points with which corresponding values are associated, determined from the reception signal.

The digitization of the measurement signals can be done at a frequency such that the digitization pitch is fine enough to subsequently perform an offset compensating for the path differences of the ultrasonic waves for the different measurement signals. This digitization frequency should then be of at least 10 times the emission frequency, namely for example 200 MHz for an emission frequency of 20 MHz, preferably at least 12 to 15 times the emission frequency in order to process the entire bandwidth of the signal in reception, which often goes up to 1.5 times the emission frequency.

These high digitization frequencies can lead to complicated digitizers especially for transducers using high-frequencies, such as 50 Mhz or more. It is to avoid these problems that, alternatively, it is also possible to add discrete points to each measurement signal by convolving said measurement signal with a sliding cardinal sine function so that a period between the discrete points is less than the inverse of at least ten times the emission frequency, and preferably less than 12 to 15 times the emission frequency.

Once the measurement signals are obtained either by direct digitization or by addition of extra points, a selection of discrete points can be selected on each measurement signal to restrict the measurement signal to these selected discrete points. The discrete points are selected so as to make a chronological offset between the different measurement signals compensating for the acoustic path differences resulting from the geometry of the transducer rings 2, the synchronism of the discrete points taking into account this offset.

Indeed, the ultrasonic waves are emitted and received by different transducer rings 2 disposed at different positions. This results in acoustic path differences resulting in time offsets. As an example, Table 1 below shows the absolute delay in nanoseconds affecting the ultrasonic waves during their path by a focal point at 15 mm in depth in the axis of their central point according to the rings in emission and in reception for an emission frequency at 20 Mhz and an ultrasonic probe of 9 mm in diameter (diameter of the outer transducer ring 2a) naturally focused at 22 mm:

TABLE 1

| | Ring in reception | | | | |
|---|---|---|---|---|---|
| Ring in emission | 2e | 2d | 2c | 2b | 2a |
| ring 2e | 25 | 53 | 82 | 112 | 143 |
| ring 2d | 53 | 81 | 109 | 140 | 171 |
| ring 2c | 82 | 109 | 139 | 169 | 200 |
| ring 2b | 112 | 140 | 169 | 199 | 230 |
| ring 2a | 143 | 171 | 200 | 230 | 261 |

Obviously, the delay is all the more significant that the transducer ring 2 is away from their common center, and the outer transducer ring 2a is the most affected. The delay affecting the ultrasonic waves results in time offsets between the measurement signals of the different transducer rings.

It is this time offset between the measurement signals between the transducer rings 2 that matters to be able to make use of the measurement signals coming from different transducer rings 2. By taking the example above and taking as a reference the measurement signal of the central annular transducer 2e for an emission by this central annular transducer 2e, the time offsets affecting the other measurement signals are given by the Table 2:

TABLE 2

| | Ring in reception | | | | |
|---|---|---|---|---|---|
| Ring in emission | 2e | 2d | 2c | 2b | 2a |
| ring 2e | 0 | 28 | 57 | 87 | 118 |
| ring 2d | 28 | 56 | 84 | 115 | 146 |
| ring 2c | 57 | 84 | 114 | 144 | 175 |
| ring 2b | 87 | 115 | 144 | 174 | 205 |
| ring 2a | 118 | 146 | 175 | 205 | 236 |

This time offset between the measurement signals results, after digitization, in an offset in number of discrete points. Thus, by using the example above, the point offsets for a digitization with a step of 2 ns is given in Table 3:

TABLE 3

| | Ring in reception | | | | |
|---|---|---|---|---|---|
| Ring in emission | 2e | 2d | 2c | 2b | 2a |
| ring 2e | 0 | 14 | 29 | 44 | 59 |
| ring 2d | 14 | 28 | 42 | 58 | 73 |
| ring 2c | 29 | 42 | 57 | 72 | 88 |
| ring 2b | 44 | 58 | 72 | 87 | 103 |
| ring 2a | 59 | 73 | 88 | 103 | 118 |

This point offset of the measurement signals must therefore be taken into account in order to match the information contained therein, which can be done simply through the selection of the discrete points of each measurement signal. For example, xₜ₁ is a discrete point of a measurement signal of a first transducer ring 2 corresponding to the instant t₁. yₜ₁ is a discrete point of a measurement signal of a second transducer ring 2 corresponding to the instant t₁. However, xₜ₁ and yₜ₁ do not take into account the same ultrasonic waves. Indeed, because of their arrangement on the ultrasonic probe, the sound waves arriving on the second transducer ring 2 have a longer path to travel and arrive with a delay d with respect to their arrival on the first transducer ring 2. Consequently, it is the point y_{t1+d} that corresponds to the same ultrasonic waves as the point xₜ₁. If the points xₜ₁, xₜ₂, xₜ₃, . . . are selected for the first measurement signal, the points y_{t1+d}, x_{t2+d}, x_{t3+d}, . . . are selected for the second measurement signal. It is thus possible to take into account the offset between the measurement signals in a simplified manner, without having to implement a time-adjustment of the measurement signals. This simplicity allows implementing this consideration of the offset practically in real time.

### Combination of the Measurement Signals

The measurement signals of the different transducer rings are then combined to give an echographic line. This echographic line is representative of the response of the transducer rings 2 at the emission of ultrasonic waves by the group of transducer rings that emitted them during this iteration. The combination of measurement signals to give an echographic line during an iteration consists in adding the values associated with discrete points of said measurement signals, with an offset corresponding to the respective delay affecting each measurement signal. By taking into account the above example, the discrete point xₜ₁ of a measurement signal of a first transducer ring 2 is added with the discrete point y_{t1+d} of another measurement signal of a second transducer ring 2, the two points being synchronous at the instant t₁ once accounted the delay d affecting the measurement signal of the second transducer ring 2 with respect to the measurement signal of the first transducer ring 2. The echographic line can of course undergo various conventional processing operations such as filtering operations, offsets or a scaling.

When enough discrete points are disposed in each measurement signal, that is to say with a frequency greater than at least 10 times the emission frequency, this then results in that an offset in number of discrete points, as explained above, corresponds to the theoretical delays affecting a measurement signal. Since the delays are no longer times, but an offset in the choice of the points in the measurement signals, it is possible to combine the measurement signals by adding them practically in real time. The echographic line can of course undergo various conventional processing operations such as filtering operations, offsets or a scaling.

### Combination of the Echographic Lines

At each iteration, the steps above are reiterated, while however modifying the group of transducer rings 2 emitting the ultrasonic waves. An echographic line is therefore obtained at each iteration. However, an echographic line represents the response of the transducer rings 2 only at the emission of ultrasonic waves by the only transducer rings 2 of the group of ultrasonic rings involved in the iteration.

It is therefore planned to combine the k echographic lines resulting from the most recent k iterations into a displayable line. The echographic lines are defined as chronological sequences of discrete points with which corresponding values are associated, and the combination of ultrasonic lines to give a displayable line consists in adding the values associated with synchronous discrete points of said echographic lines. It should be noted that the combination of these echographic lines can be performed as soon as said echographic lines are available. Consequently, the combinations can be made in parallel with the continuation of the cycles following the first cycle.

The k combined echographic lines can result from the k iterations of a cycle if the latter has just ended, or from the last k-i iterations of one cycle and the i iterations of the next cycle, with l<i<k. For example, with reference to FIG. 2, the iterations 101, 102, 103, 104, 105 of the first cycle 100 each give an echographic line (k=5). A first combination 106 relates to the echographic lines resulting from the iterations 101, 102, 103, 104, 105 of the first cycle 100. The second combination 107 relates to the last four echographic lines of the first cycle 100, that is to say resulting from the iterations 102, 103, 104, 105, and on the first echographic line of the second cycle 110, that is to say resulting from the iteration 111. The third combination 108 relates to the last three echographic lines of the first cycle 100, that is to say resulting from the iterations 103, 104, 105, and to the first two echographic lines of the second cycle 110, that is to say resulting from the iterations 111 and 112. The following combinations take place in a similar way and so on, by offsetting one iteration for each new displayable line.

For example, with reference to FIG. 3, the iterations 201, 202, 203 of the first cycle 200 each give an echographic line (k=3). A first combination 204 relates to the three echographic lines resulting from the iterations 201, 202, and 203 of the first cycle 200. The second combination 205 relates to the last two echographic lines of the first cycle 200, that is to say resulting from the iterations 202 and 203, and to the first echographic line of the second cycle 210, that is to say resulting from the iteration 211. The third combination 206 relates to the last echographic line of the first cycle 200, that is to say resulting from the iteration 203, and to the first two echographic lines of the second cycle 210, that is to say resulting from the iterations 211 and 212. The following combinations take place in a similar way and so on, by offsetting one iteration for each new displayable line.

For example, with reference to FIG. 4, the iterations 301 and 302 of the first cycle 300 each give an echographic line (k=2). A first combination 303 relates to the two echographic lines resulting from the iterations 301 and 302 of the first cycle 300. The second combination 304 relates to the last echographic line of the first cycle 300, that is to say resulting from the iteration 302, and to the first echographic line of the second cycle 310, that is to say resulting from the iteration 311. The third combination 305 relates to the two echographic lines resulting from the iterations 311 and 312 of the second cycle 310. The following combinations take place in a similar way and so on, by offsetting one iteration for each new displayable line.

A sliding combination is thus made of the last k echographic lines resulting from the most recent k iterations to obtain each of the displayable lines. To display N displayable lines on a screen, N+k−1 iterations are then carried out. For example, to display 400 displayable lines, 404 iterations are carried out if k=5, which represents a negligible overhead in iterations. In order to make the digital lines of the displayable signal and thus produce an image, it is of course possible to make them undergo various conventional processing operations such as filtering, rectification, offset, and logarithmic scaling.

With this approach, it is possible to modify in real time the emission modes of the ultrasonic waves, by modifying the composition of the groups of rings, for example by moving from groups of one transducer ring 2 to groups of three transducer rings 2. It is also possible to modify the processing modes of the measurement signals, by changing the delays affecting them during their combination. Depending on the chosen medical target, it is thus possible to promote the resolution, sensitivity, penetration or speed by using different emission or reception configurations.

For example, by using groups of rings consisting of a single transducer ring 2 as in FIG. 1, the best compromise between the resolution, penetration and sensitivity on the overall image of an eye is obtained.

With this approach, the speed is substantially increased compared to the phase-controlled conventional approach, since at each cycle or iteration, the entire depth inspected is examined. There is therefore no more need to make multiple passages in the same location to image at different depths. Compared to the radar type approach, a displayable line by iteration is obtained after the first cycle, which allows being much faster since the radar type approach produces only one displayable line per cycle. A speed almost similar to a mono-transducer is then reached, which is fundamental in ophthalmology, in particular because of the rapid movements of the eye.

A unit for automated processing of data comprising at least one processor and a memory is used for the processing of the image data, and in particular for combining the measurement signals or the lines.

The invention is not limited to the embodiment described and represented in the appended figures. Modifications remain possible, in particular from the point of view of the constitution of the various elements or by substitution of technical equivalents, without departing from the field of protection of the invention.

1. An ocular echography method using an ultrasonic probe including a plurality of transducer elements organized into at least n concentric rings forming n transducer rings, wherein the transducer rings are grouped into several groups of rings each grouping between 1 and n−1 transducer rings, each group of rings being differentiated from another group of transducer rings by at least one transducer ring different from the transducer rings of said other group of transducer rings, the groups of rings being equal to a number k, the method comprising the following steps:
a) for each cycle of a plurality of cycles of k iterations, each iteration involving a group of different transducer rings, each cycle running through the set of the k groups of transducer rings, during each iteration:
a1) exciting a group of transducer rings so that the transducer rings of said group of transducer rings emit ultrasonic waves at an emission frequency;
a2) recovering n measurement signals from the n transducer rings, each measurement signal resulting from the reception by a transducer ring of reflected ultrasonic waves resulting from the emission of ultrasonic waves by said group of transducer rings;
a3) combining the n measurement signals to give an echographic line, said echographic line being representative of the response of the n transducer rings to the emission of ultrasonic waves by said group of ultrasonic rings;
b) combining into a displayable line of k echographic lines resulting from the most recent k iterations;
c) processing and displaying the displayable lines.

2. The method according to claim 1, wherein in each cycle, the iterations are performed in the same order.

3. The method according to claim 1, wherein the measurement signal of a transducer ring results from the digitization of the reception signal generated by this transducer ring upon reception by said transducer ring of reflected ultrasonic waves resulting from the emission of ultrasonic waves by a group of transducer rings, a measurement signal being defined as a chronological sequence of discrete points with which corresponding values are associated,
and wherein the combination of measurement signals to give an echographic line during an iteration consists in adding the values associated with synchronous discrete points of said measurement signals.

4. The method according to claim 3, wherein the combination of measurement signals to give an echographic line during an iteration is restricted to a selection of discrete points, said discrete points being selected so as to make a chronological offset between the measurement signals compensating for the acoustic path differences resulting from the geometry of the transducer, the synchronism of the discrete points taking into account this offset.

5. The method according to claim 3, wherein discrete points are added to the measurement signal by convolving said measurement signal with a sliding cardinal sine function so that a period between the discrete points is less than the inverse of at least ten times the emission frequency.

6. The method according to claim 1, wherein an echographic line is defined as a chronological sequence of discrete points with which corresponding values are associated, and wherein the combination of echographic lines to give a displayable line consists in adding the values associated with synchronous discrete points of said echographic lines.

7. The method according to claim 1, wherein the transducer rings are grouped into k groups of rings each grouping between 2 and n−1 transducer rings.

8. The method according to claim 1, wherein n=5, the ultrasonic probe comprising five transducer rings.

9. A non-transitory computer-readable medium comprising program code instructions recorded thereon that causes a computer to perform the steps of the method according to claim 1, when said medium is read by a computer.

10. An echography system comprising an ultrasonic probe including a plurality of transducer elements organized into at least n concentric rings forming n transducer rings, a unit for processing said ultrasonic probe and a screen, said processing unit being configured to implement the method according to claim 1, the transducer rings being grouped into several groups of rings each grouping between 1 and n−1 transducer rings, each group of rings being differentiated from another group of transducer rings by at least one transducer ring different from the transducer rings of said other group of transducer rings, the groups of rings being equal to a number k, the processing unit being configured to:
a) for each cycle of a plurality of cycles of k iterations, each iteration involving a different group of transducer rings, each cycle travelling through the set of the k groups of transducer rings, during each iteration:
a1) excite a group of transducer rings so that the transducer rings of said group of transducer rings emit ultrasonic waves at an emission frequency;
a2) recover n measurement signals from the n transducer rings, each measurement signal resulting from the reception by a transducer ring of reflected ultrasonic waves resulting from the emission of ultrasonic waves by said group of transducer rings;
a3) combine the n measurement signals to give an echographic line, said echographic line being representative of the response of the n transducer rings to the emission of ultrasonic waves by said group of ultrasonic rings;
b) combine into a displayable line of k echographic lines resulting from the most recent k iterations;
c) process and send back the displayable lines to the screen.
